(19) United States
(12) Patent Application Publication
(10) Pub. No.: US 2021/0283095 A1
(43) Pub. Date: Sep. 16, 2021

(54) Detection and Treatment of Viral Diseases and Cancer

(71) Applicant: Harold E. Siess, Woodstock, GA (US)

(72) Inventor: Harold E. Siess, Woodstock, GA (US)

(21) Appl. No.: 17/249,734

(22) Filed: Mar. 11, 2021

Related U.S. Application Data
(60) Provisional application No. 62/987,912, filed on Mar. 11, 2020.

(51) Int. Cl. A61K 31/34; A61K 45/06

(52) U.S. Cl. CPC A61K 31/34; A61K 45/06

(57) ABSTRACT

A method for treating unwanted cells in a patient by administering a first substance that is capable of forming a dehydroascorbic acid-like substance to the patient, thereby forming a pool of reactive molecules within the unwanted cells, and administering a second substance for reacting with the pool of reactive molecules within the unwanted cells, thereby forming a product within the unwanted cells.

### PRIORITY

This application is a non-provisional of, and claims rights and priority on, prior pending U.S. provisional patent application Ser. No. 62987912 filed Mar. 11, 2020, the entirety of the disclosure of which is incorporated herein by reference.

### FIELD

This invention relates to the field of medicine. More particularly, this invention relates to the detection and treatment of viral diseases and cancer.

### INTRODUCTION

For a variety of different reasons, humans tend to develop certain kinds of unwanted cells. For example, some of these unwanted cells include diseased cells, such as virally-infected cells, and proliferative cells, such as cancer cells. It can be difficult to detect the presence of such cells, and then, even if detected, it can be difficult to treat such cells.

What is needed, therefore, is a method that tends to reduce issues such as those described above, at least in part.

### SUMMARY

The above and other needs are met by a method for treating unwanted cells in a patient by administering a first substance that is capable of forming a dehydroascorbic acid-like substance to the patient, thereby forming a pool of reactive molecules within the unwanted cells, and administering a second substance for reacting with the pool of reactive molecules within the unwanted cells, thereby forming a product within the unwanted cells.

In various embodiments according to this aspect of the invention, the product includes at least one of a sensitizing agent, a tracer agent, and a therapeutic agent. In some embodiments the second substance includes metal ions that form the product with a cellular component of the unwanted cells, the cellular component in the form of at least one of sulfur-containing molecules and selenium-containing molecules. In some embodiments the second substance includes halogen ions that form the product in the form of halogen molecules. Some embodiments include passing EMR through the product within the unwanted cells. In some embodiments the second substance is at least one of radioactive gold, radioactive silver, radioactive iodine ions, nonradioactive gold, nonradioactive silver, and nonradioactive iodine ions. In some embodiments the unwanted cells are pathologically-altered cells having upregulated glucose transporters. In some embodiments the unwanted cells include at least one of a pathological formation of proliferative cells, a cancerous tumor, proliferative smooth-muscle cells associated with hyperplasia of pulmonary artery smooth-muscle cells, cell types that produce an unwanted amount of fibrogenic materials, cells that have been invaded by an infective agent, and hyper-activated immune cells that cause hyper-inflammation. Some embodiments include administering a GLUT-1 blocker to the patient to prevent passage of glucose and vitamin C into the unwanted cells, thereby potentiating the unwanted cells to radiation-based therapies by the entrapment of dehydroascorbic acid therein.

According to another aspect of the invention there is described a method for classifying test cells by applying first metallic ions to healthy cells, irradiating the healthy cells, and recording properties of secondary emissions from the healthy cells. The method continues by applying second metallic ions to pathogenic cells, irradiating the pathogenic cells, and recording properties of secondary emissions from the pathogenic cells. The method continues by applying third metallic ions to the test cells, irradiating the test cells, recording properties of secondary emissions from the test cells, and comparing the properties of the test cells secondary emissions to the healthy cells secondary emissions and the pathogenic cells secondary emissions to determine whether the test cells are healthy or pathogenic.

Some embodiments according to this aspect of the invention further include the steps of applying a first substance that can form a metal-reducing agent within cells to the healthy cells prior to irradiating the healthy cells, applying a second substance that can form a metal-reducing agent within cells to the pathogenic cells prior to irradiating the pathogenic cells, and applying a third substance that can form a metal-reducing agent within cells to the test cells prior to irradiating the test cells.

In some embodiments the first metallic ions, second metallic ions, and third metallic ions are silver. In some embodiments the first substance, second substance, and third substance are vitamin C. In some embodiments the applying and irradiating steps are performed in at least one of in-vitro, in-vivo, a subject's throat, and the subject's respiratory system. In some embodiments the pathogenic cells are virus-infected cells. In some embodiments the pathogenic cells are proliferative cells. In some embodiments the pathogenic cells are cancer cells.

### DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a flowchart of a generalized method for selectively forming and using a tracer for at least one of treating a subject with a pathogenic condition and detecting the presence of a pathogenic condition within a subject, according to an embodiment of the present invention.

FIG. 2 is a flowchart of a depositional method for analysis of detecting, locating, and characterizing a pathogenic entity within a subject, according to an embodiment of the present invention.

FIG. 3 is a flowchart of an in vivo method for detecting the presence of a viral infection, according to an embodiment of the present invention.

FIG. 4 is a flowchart of an in vitro method for detecting the presence of a viral infection in a subject using emission based methods, according to an embodiment of the present invention.

FIG. 5 is a flowchart of an in vitro method for detecting the presence of a viral infection in a subject using vitamin C enhanced emission based methods, according to an embodiment of the present invention.

FIG. 6 is a flowchart of a method for treatment of a patient suffering from proliferative disorder, wherein it is preferable that the pathogenicly affected cells be destroyed by radiological energy, according to a first embodiment of the present invention.

FIG. 7 is a flowchart of a method for treatment of a patient suffering from proliferative disorder, wherein it is preferable that the pathogenicly affected cells be destroyed by radiological energy, according to a second embodiment of the present invention.

FIG. 8 is a flowchart of a method for treatment of a patient suffering from at least one of a proliferative disorder and an intracellular infection, wherein it is preferable that the pathogenically affected cells not be destroyed, according to an embodiment of the present invention.

### DESCRIPTION

### Theoretical Bases

This section provides theoretical bases for the embodiments of the present invention. However, the embodiments are provided hereafter with being limited by any theory.

A number of proliferative cell types, such as cancerous cells, undergo the Warburg Effect and upregulate glucose transporters, in particular the GLUT-1 transporter. These cell types are generally referred to as pathogenic cells herein, which term also includes, as described further herein, virally infected cells and proliferative lesions. Many virally infected cells also undergo the Warburg Effect and increase the uptake of glucose in a similar fashion as cancer cells through GLUT-1 and GLUT-4 transporters. The corona virus also produces the Warburg Effect in the infected epithelial cells lining the respiratory tract. Both cancer cells and virally infected cells increase the consumption of glucose as a way of increasing both the rate of replication of DNA or RNA and the production of proteins and the like. It also appears that the corona virus produces the Warburg Effect in infected epithelial cells.

The glucose transporters are the main mechanism by which dehydroascorbic acid (DHA) enters cells, particularly the GLUT-1 transporter. The transport of DHA by this mechanism is applicable to both cells containing GLUT-1 as a natural constituent as well as for cells where GLUT-1 transporters have been pathologically upregulated. Once inside the cell, DHA is reduced back into ascorbate by one or more of the reductive mechanisms within the cell that recycle vitamin C. In most cell types there is no mechanism by which ascorbic acid can efflux from the cell. For these non-ascorbic acid effluxing cell types the ascorbic acid within the cell is trapped. Therefore, ascorbic acid can accumulate within these types of cells up to concentrations far exceeding that of the extracellular fluid in which they are contained. By this method of uptake and reduction, the ascorbic acid concentration in the cytosol of some cell types reaches millimolar levels that are 10-100 times greater than the ascorbic acid concentration in the surrounding extracellular fluids.

The degree to which dehydroascorbic acid is transported into the cell depends significantly upon the pH of the extracellular fluid. In those cell types that are surrounded by an acidic medium ascorbic acid can be readily oxidized within the extracellular fluid and transported into the cell as dehydroascorbic acid.

Many cancerous formations can be detected using F18 labeled glucose. However, F18 labeling of dehydroascorbic acid can also be used to detect cancerous cells within the body having upregulated GLUT-1 transporters. 14C-labeled ascorbate can also be used to analyze the uptake and conversion of vitamin C within erythrocytes and other cell types that have been incubated in DHA-containing solutions.

Of interest are the effects of the upregulation of the transporters that transport glucose into benign, hyperplasic, preneoplastic and premalignant lesions and cancer cells. For example, the expression of GLUT-1 in fibroblasts is higher in proliferating cells when compared to contact-inhibited ones. Of interest is the role of GLUT transporters in supplying vitamin C.

Pulmonary arterial hypertension (PAH) is a progressive disorder characterized by narrowing of the pulmonary arteries due to muscular thickening and invasive proliferation of smooth muscle cells (SMC) into the intima and into multiplex regions of the blood vessel. The hyperplasia of pulmonary artery smooth-muscle cells (PA-SMCs) is the main pathogenic change responsible for the vascular remodeling observed in this disease. The activated neutrophils involved in cystic fibrosis also have an upregulation of the GLUT-1 transporters.

Intracelluar pathogens upregulate GLUT-1 transporters. Metabolic phenotypes conferred by virus infection often mirror metabolic changes seen in cancer cells, such as upregulation of glucose transporters to support viral replication or rapid cell growth, respectively. Most viral infections, such as the dengue virus, HBV, EBV, Kaposi's sarcoma-associated herpesvirus, and the Merkel cell polyomavirus, upregulate the glucose transporter 1 (GLUT-1). However, HCMV downregulates the ubiquitously-expressed glucose transporter-1 and concurrently increases the expression of GLUT-4 to accelerate glucose uptake.

Influenza A virus increases glycolysis, enhancing glucose uptake and lactate production at early time points post-infection, as well as increases oxygen consumption rates. The type of transporter used to increase the uptake of glucose has not been reported.

18F-FDG PET/CT in patients with acute respiratory disease caused by COVID-19 in Wuhan, Hubei province of China were determined. Lung lesions were characterized by a high 18F-FDG uptake and there was evidence of lymph node involvement. Conversely, disseminated disease was absent.

Positron-emission tomography (PET) is a nuclear medicine functional imaging technique that is used to observe metabolic processes in the body as an aid to the diagnosis of disease, typically cancer. The system detects pairs of gamma rays emitted indirectly by a positron-emitting radioligand, most commonly fluorine-18, which is introduced into the body on a biologically active molecule called a radioactive tracer.

Tumors can be imaged using radioactive tracers in the form of 6-deoxy-6-[18F]fluoro-L-ascorbic acid and/or 6-[18F]Fluoro-Dehydroascorbic Acid and/or 6-Deoxy-6-[131I]iodo-L-ascorbic acid for PET scan imaging.

Photoacoustic (PA) imaging is an emerging medical imaging method that combines optical excitation of contrast agents and ultrasound detection. This form of imaging is used to generate high-resolution images at centimeters depth. Wavelengths in the second near-infrared (NIR-II) window (1000 to 1700 nm) have preclinical and clinical applications. The NIR-II window offers numerous advantages, including high spatial resolution, deeper penetration depth, reduced optical absorption, and tissue scattering. Moreover, the second window allows a fivefold higher light excitation energy density compared to the visible window for enhancing the imaging depth significantly. There is also importance for the second window for PA imaging and the various NIR-II PA imaging systems and contrast agents with strong absorption in the NIR-II spectral region.

NIR-II (second near-infrared) emitting agents for in-vivo imaging applications include QDs such as PbSe, PbS, and CdHgTe, with NIR emission. But, the highly toxic nature of Pb, Cd, and Hg is of concern for in-vivo applications. Ag2S QDs have high-emission efficiency in the unique NIR-II imaging window. So, there are a lot of characteristics such as deep tissue penetration, high sensitivity, and elevated spatial and temporal resolution that make Ag2S Qds useful as imaging agents.

The cortical portion of ox suprarenals turn black when immersed in neutral silver nitrate solution. The compound, ascorbic acid, responsible for the reduction can be isolated in crystalline form from ox suprarenals. The feeding of orange juice to scorbutic animals is followed by the prompt reappearance of ascorbic acid in the cortex of the adrenals.

After deposition, silver can be oxidized to silver sulfide or silver selenide, resulting in blue-gray pigmentation. Silver selenide is highly insoluble in aqueous solutions nor is it readily solubilized by any physiological mechanisms; therefore, it is poorly absorbed after exposure and is more likely to be excreted by the body.

Silver is used as an antimicrobial agent. A fundamental property enabling silver metal ions to be applied against invading pathogens is their low toxicity in uninfected mammalian cells. In contrast to mammalian cells, silver ions damage pathogens by reacting with nucleic acids, cell wall components, cysteine molecules or sulfhydryl groups of key metabolic enzymes. In the reaction with enzymes, ionic silver combines with the sulfhydryl groups forming hemisilver sulfides. The denatured protein precipitates out of solution.

While ionic silver primarily reacts with proteins, it is also known to react with RNA and DNA, by binding to amino, carboxyl, phosphate and imidazole groups.

Metals attack a broad range of targets during viral infection and, as such, offer a lower possibility for the development of resistance as compared to conventional antivirals. Silver nanoparticles are active against several types of viruses including human immunodeficiency virus, hepatitis B virus, herpes simplex virus, respiratory syncytial virus, and monkey pox virus.

Radiotherapy provides a locoregional method of treatment that may be used in conjunction with both (1) other localized methods, such as heating, and (2) systemic treatments using chemotherapy. The success of radiotherapy is impaired by four of the following processes: (1), the pretreatment radioresistance of the tumor cells to the applied doses, (2), the development of cancer stem cells having a more radioresistant phenotype after exposure to radiotherapy by an increased capacity for DNA-repair and for scavenging of reactive oxygen species as seen in breast cancer, glioblastoma and lung cancer, (3), the radiation-induced damage of uninfected tissue located both inside and outside the applied beam of ionizing radiation and (4), the lack of efficient mechanisms to use radiation-based therapies in conjunction with chemotherapy or thermotherapy.

Methods can be used to overcome the problems associated with radiation-based therapeutics. Two techniques to specifically target unwanted cell types while saving uninfected cell types are (1) radiosensitization of the tumor cells without sensitizing uninfected tissue cells; and (2) selective radioprotection of uninfected tissue cells as a way of overcoming the problem of a limited dosing regimen. Therefore, a major goal in developing radiotherapeutic methods is the development of agents that selectively increase the sensitivity of tumor cells or decrease the sensitivity of uninfected tissues to the effects of ionizing radiation.

One type of sensitizer is the photothermal-sensitizer. Photothermal-sensitizers are in the form of metallic nanoparticles that are injected into the body of the individual to be treated. Photothermal-sensitizer materials differ from other types of radiosensitizers in that they are also able to selectively absorb electromagnetic radiation at frequency below that of x-rays. They can therefore be used to selectively heat cancerous cells by absorbing microwave radiation for example.

In the first step in the therapeutic use of photothermal-sensitizers, a beam of electromagnetic waves (EMR) from a radiation source is used to selectively excite the photothermal-sensitizing agent within the tumor. The beam of electromagnetic radiation can be in the form of radiofrequency waves and/or microwaves from an appropriate source. The excited photothermal-sensitizer agent releases heat into the tumor for killing unwanted cell types. One type of photothermal-sensitizer under development uses gold nano-shell particles with 100-nm silica cores and a 15-nm gold coating, which have a resonance peak in the near infrared region (650-950 nm) where blood and tissue are maximally transmissive for selectively heating the tumor by the use of IR. Ionizing radiation, which can be in the form of x-rays, can also be used simultaneously to excite photothermal-sensitizers for creating additional secondary energies such as short-range low-energy electrons, Auger electrons, photoelectrons or characteristic X-rays and/or heat that are used to drive the production of radicals. The major problem associated with this form of IRB therapeutics is the selective placement of the nanoparticles within the tumor and the efficient production of oxidative free radicals from the free electrons released from the metal.

Mild temperature hyperthermia generally refers to temperatures between 40° C. and 45° C. This form of treatment mediates its antitumor effects via subtle influences on the tumor microenvironment, induction of apoptosis, activation of immunological processes, and induction of gene and protein synthesis. The rise in temperature does not independently cause tumor cell cytotoxicity, but rather lead to greater effectiveness of other conventional treatment modalities such as RT, chemotherapy, and immunotherapy. In its role as an adjunct to RT, hyperthermia serves as a dose-modifying agent that increases the therapeutic ratio of RT (i.e., enhanced effectiveness of a given dose of RT without additional toxicity).

There are several problems associated with using absorption based radiosensitizing agents. Perhaps the most important of these problems is the toxicity of these types of agents. For example, the use of iopamidol as a radiosensitizer is known to cause heart attack, stroke, and blood clotting problems or severe kidney problems. Another significant problem associated with the use of radiosensitizers and their methods of application is the difficulty of selectively placing these agents within tumors.

### Specific Embodiments

GLUT-1 transporters transport the oxidized form of ascorbic acid, which is called dehydroascorbic acid (DHA). Once inside the cell, DHA is rapidly converted back to ascorbic acid. For most cells in the body there is no cellular mechanism to efflux the excess ascorbic acid and, therefore, during the rapid transport of DHA into the cell, a pool of excess ascorbic acid can form in the cell. During viral infections the patient is under oxidative stress and therefore a large amount of DHA is formed within the blood. Therefore, virally infected cells having upregulated GLUT-1 transporters tend to uptake a large amount of DHA, which is reduced to ascorbic acid within the cell.

In various embodiments, pools of ascorbic acid are used to reduce silver ions into particulate matter comprised of silver sulfide particles. The formed silver sulfide particles are used to at least one of (1) detect the presence of cells that have pathogenicly-upregulated transporters of either ascorbic acid or DHA, (2) kill the pathogenic cells, and (3) allow the silver sulfide particles formed within the pathogenic cells to absorb selenium and thereby reduce the levels of selenium-containing proteins needed to replicate either cancer cells or viruses within cells as a way of therapeutically treating the pathogenic cells, while still maintaining their primary functional roles. In the case of the viral infections that upregulated GLUT-1 transporters within the respiratory track, the silver-containing particles also act as a source of Ag+ that helps to fight off the secondary bacterial infections that are known to eventually lead to the death of the patient suffering from infections, such as produced by the novel corona virus.

Some embodiments provide a method and composition for at least one of detecting and therapeutically treating unwanted cells, such as infected cells, particularly virally infected cells, pathogenicly proliferative cells, such as but not limited to cancerous cells, and those cell types that produce an unwanted amount of fibrogenic materials, again generally referred to herein as pathogenic cells. Some embodiments describe a method and composition for detecting the level in the metabolic activities of at least one of ascorbic acid and dehydroascorbic acid for determining the presence of a pathogenic condition and treating the subject with the method and composition.

Some embodiments describe a method for at least one of detecting and treating pathogenic cells having a pathogenicly-upregulated transporter of vitamin C for at least one of (1) detecting the presence of cells that have pathogenicly-upregulated transporters of either ascorbic acid or DHA, (2) killing the pathogenicly altered cells, and (3) allowing the silver sulfide particles formed within the cells to act as a getter for selenium and thereby reduce the levels of selenium-containing proteins needed to replicate either cancer cells or viruses within cells as a way of therapeutically treating the pathogenic cells, while still maintaining their primary functional roles.

Some embodiments describe a method and composition for selectively forming and using a sensitizer to detect and therapeutically treat human pathogenic cells, which may be in the form of cells that have pathogenicly-upregulated transporters of vitamin C or derivatives thereof, with the cells being comprised of at least one of (a) a pathogenic formation of proliferative cells, such as, for example, a cancerous tumor or the proliferative smooth-muscle cells associated with hyperplasia of pulmonary artery smooth-muscle cells, (b) those cell types that produce an unwanted amount of fibrogenic materials, and (c) cells that have been invaded by an infective agent, by the selective deposition of metallic substances within the pathogenic cells for forming particle enriched cells for either treating pathogenic cells in a subject with a pathogenic condition or for detecting the presence of a pathogenic condition comprising the steps of (A) exposing the pathogenic cells to a substance that is capable of forming a reducing agent within the pathogenic cells for forming a pool of reducing agent, which may be comprised of ascorbic acid or derivative thereof, therein; (B) exposing the pathogenic cells to a substance which may be in the form of ions comprised of metals, which may be in the form of radioactive metallic ions such as 111Ag+ and 112Ag+ ions, for undergoing an oxidation reduction reaction with the pool of reducing agent, for forming an exogeneous substance that may be comprised of solid particulates comprised of a reduced form of the substance which may be comprised of a metal for either (1) sensitizing the selenium within the pathogenic cells to precipitate out of solution for reducing the levels of selenium containing enzymes within the pathogenic cells for reducing the proliferation rates occurring within the pathogenic cells for treating a subject with a pathogenic condition and (2) acting as a stain for detecting the presence of a pathogenic condition by either (a) the emission of secondary waves, which may be in the form of electromagnetic or acoustic wave, from the particulates or (a) by the attenuation in the beam of electromagnetic radiation passing through the exogeneous substance or (3) sensitizing the altered cells to at least one of the following, (a) electromagnetic radiation, which may be in the form of X-rays, IR or microwaves, or (b) charged particles, or (c) reacting with radioactive atoms, which may be in the form of radioactive selenium atoms, such as 75Se, or 72Se or (d) chemotherapeutic agents. The method for forming a sensitizer within aberrant cells is particularly useful for at least one of (1) sensitizing at least one unwanted cell types to the effects of X-rays, IR or microwaves or chemotherapy and (2) tracing at least one of the appearance and location of the pathogenic cells.

In another embodiment, the method for determining the presence of a viral infection includes the steps of (a) taking a biological sample from a subject, such as from the back of the throat, and (b) detecting the upregulation of at least one glucose transporters using immunofluorescence microscopy. However, the method for determining the presence of infection by the formation of metallic deposits using upregulated glucose transporters can be (1) quickly made commercially available, (2) will give a better determination of the exact type of infection present, and (3) can be done using in vitro techniques.

Another embodiment includes the steps of administering metallic nanoparticles that target pathogenic cells, administering a reactive radioactive substance, such as selenium, that combine with the nanoparticles to form radioactive nanoparticles and kill the pathogenic cells.

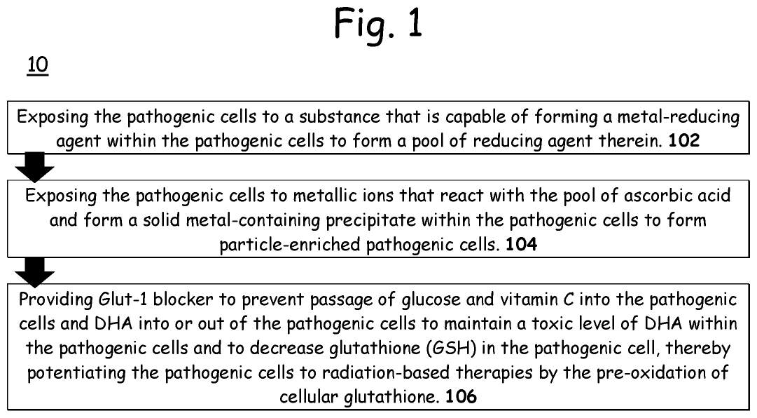

With reference now to FIG. 1, there is depicted a flow chart of a generalized method 10 for detecting and therapeutically treating pathogenic cells, which may be in the form of at least one pathogenicly-altered human cells having upregulated transporters of vitamin C or a derivative thereof, and which may be comprised of at least one of (a) a pathogenic formation of proliferative cells, such as, for example, a cancerous tumor or the proliferative smooth-muscle cells associated with hyperplasia of pulmonary artery smooth-muscle cells, (b) those cell types that produce an unwanted amount of fibrogenic materials, and (c) cells that have been invaded by an infective agent, by the selective deposition of a metal containing tracer within the pathogenicly-altered cells for forming particle enriched cells for at least one of treating a subject with a pathogenic condition and detecting the presence of a pathogenic condition within a subject.

As given in block 102, the altered cells are exposed to a substance that is capable of forming a metal reducing agent within the altered cells for forming a pool of reducing agent therein. In one embodiment, the reducing agent is in the form of vitamin C or a derivative thereof, for forming a pool of ascorbic acid or derivative thereof within the altered cells.

As given in block 104, the altered cells containing the pool of reducing agent are exposed to metallic ions for reacting with the pool of ascorbic acid for forming a solid metal containing precipitate having a high mass attenuation coefficient or high absorption cross section within the pathogenicly altered cells for forming both particle enriched cells and the toxic products of ascorbic acid oxidation, such as, dehydroascorbic acid, or derivative thereof and decreasing the level of GSH within the cells for at least one of (1) sensitizing the selenium within the altered cell to precipitate out of solution for reducing the levels of selenium containing enzymes within the altered cells for reducing the proliferation rates occurring within the altered cells for treating a subject with a pathogenic condition, (2) sensitizing the altered cells to at least one of (a) electromagnetic radiation, which may be in the form of X-rays, IR or microwaves, (b) charged particles, and (c) radioactive atoms, which may be in the form of radioactive selenium atoms, such as 75Se, or 72Se, and (3) chemotherapeutic agents that increase the level of hydrogen peroxide within cells, for at least one of treating a subject with a pathogenic condition and detecting the presence of a pathogenic condition within a subject. In one embodiment, the metal ions are in the form of heavy metal ions, such as gold or silver.

In one embodiment for treating a subject suffering from a proliferative disorder, after the pools of reducing agents (silver deposits) have been formed, a commercially available GLUT-1 blocker is supplied to the subject for preventing the passage of glucose and vitamin C into the infected cells and to prevent the passage of DHA into or out of the cell for both maintaining a toxic level of DHA within the cell and for decreasing the level of glutathione (GSH) in the cell for potentiating the cell to radiation based therapies by the pre-oxidation of cellular glutathione, as given in block 106.

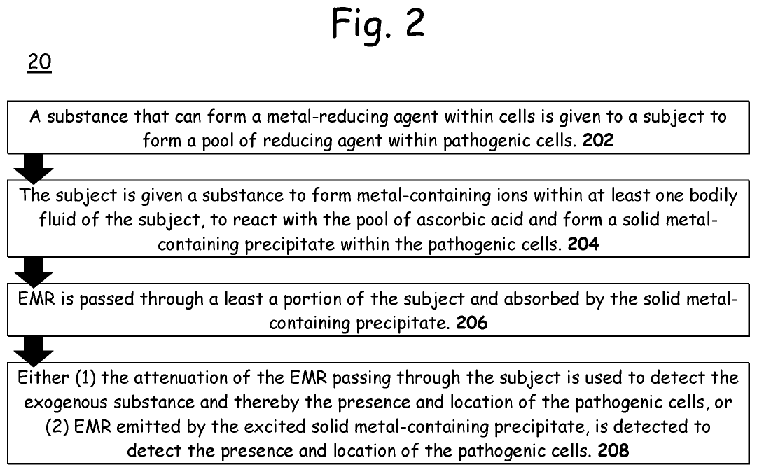

An exemplar on a method 20 for the detection of pathogenicly altered cells within a living subject using the teachings of the present invention is given in FIG. 2. With reference to FIG. 2, there is shown a flow chart of a depositional method of analysis for at least one of detecting, locating, and characterizing a pathogenic entity within a subject, which can be either human or animal, using selective deposition. In the first step of the method of the selected depositional method of analysis as given in block 202, a substance that is capable of forming a metal reducing agent within cells is provided to the subject for forming a pool of reducing agent within a pathogenic lesion (or entity), which may be in the form of (1) a pathogenic formation of proliferative cells, such as cancerous a tumor, or tissue that has been invaded by pathogenic agents such as viruses or bacteria. In one embodiment the reducing agent is in the form of vitamin C or derivative thereof for forming a pool of ascorbic acid or derivative thereof within the pathogenic lesion or entity. In one embodiment the vitamin C is administered through a non-oral route such as by intravenous injection for increasing the level of plasma vitamin C that can be obtained by oral administration. In one embodiment the vitamin C is injected by slow infusion of doses on the order of 0.1 gram to 1.0 gram per kilogram body mass for obtaining a concentration of ascorbate within the blood of over about 0.5 mM.

As given in block 204, the patient is provided a substance for forming metal containing ions within at least one bodily fluids of patient for reacting with the pool of ascorbic acid for forming a solid metal containing precipitate within the pathogenic lesion or entity. In one embodiment, the metal ions are in the form of heavy metal ions, such as gold or silver. In one embodiment the metal has a lower toxicity than that of lead ions. The ions may be formed within the general circulation by either of two administrative methods. In the first administrative method either a salt or the metal or particles of the metal in colloidal form are administered to the subject. In the second administrative method, a salt of the metal is introduced by intravenous administration. In one embodiment the concentration of silver ions within the blood is maintained below about 0.5 ppm.

As given in block 206, a beam of electromagnetic waves, which may be in the form of x-rays, infrared waves, radiofrequency waves, or microwaves, is passed through at least a portion of the subject for absorption by the solid metal containing precipitate. In one embodiment the infrared waves are in the near infrared region (from about 650 nm to about 950 nm) where blood and tissue are maximally transmissive.

As given in block 208, at least one of (1) the attenuation of the electromagnetic waves passing through target cells is used for at least one of detecting and analyzing the exogenous substance for detecting at least one of the presence and location of a pathogenic lesion, and (2) at least one waves emitted by the excited exogenous substance, which may be in the form of acoustic waves or electromagnetic waves, such as infrared waves, are detected for detecting the presence and location of a pathogenic lesion or entity.

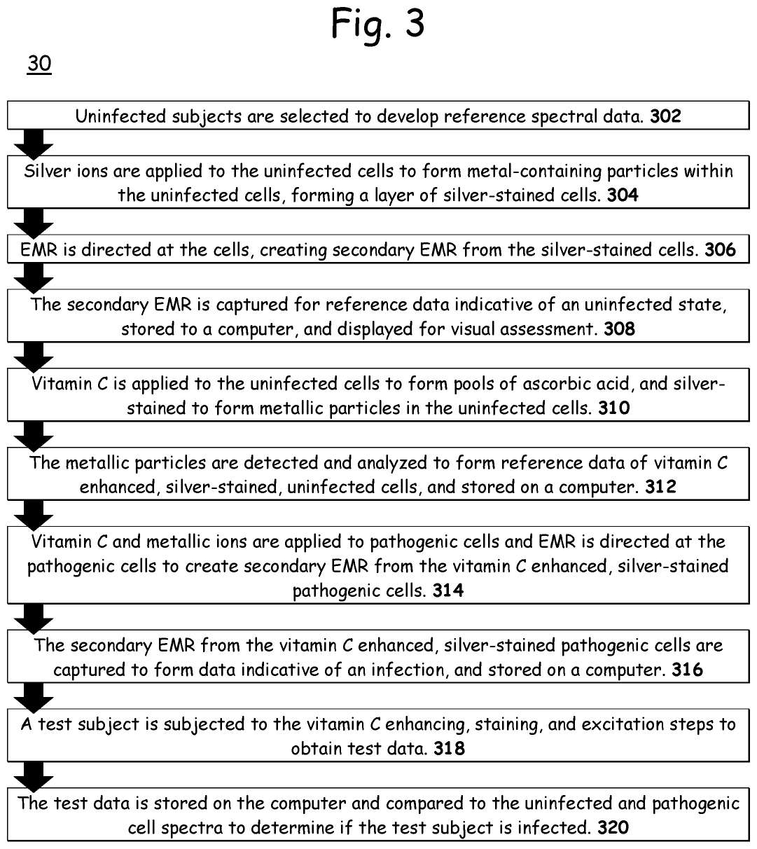

An exemplar on a method 30 for the detection of a viral infection within a living subject using an embodiment of the present invention is given in FIG. 3. With reference to FIG. 3, there is depicted a flow chart of an in vivo method for detecting the presence of a viral infection in a subject. As given in block 302, at least one uninfected subjects are selected for forming reference spectral data. As given in block 304, a solution containing metallic ions, which may be in the form of silver or gold ions, is applied to the back of the throat of the reference subjects for forming solid metal containing particles within the outer cellular layer of their throats for forming a layer of silver-stained cells.

As given in block 306, a beam of electromagnetic radiation, which may be in the form of at least one of x-rays, infrared waves, radiofrequency waves, and microwaves, is directed at the outer most cellular layer of the throats of the reference subjects for creating secondary waves, which may be in the form of electromagnetic waves such as infrared waves or acoustic waves, from the silver-stained layer of uninfected cells. As given in block 308, the secondary waves are captured for forming a first set of reference spectroscopic data that is indicative of an uninfected state. The reference spectroscopic data is sent to a device, such as a computer, for forming a set of stored reference spectroscopic data for later comparison. The reference spectroscopic data may be displayed on a screen for visually assessing the image created.

As given in block 310, a solution containing vitamin C is applied to the back of the throats of a different set of uninfected subjects for forming pools of ascorbic acid within the uninfected layer of cells. After three or more minutes, a solution of metallic ions is applied to the back of the throats for forming small metallic particles within the layer of uninfected cells. As given in block 312, the small metallic particles are detected by directing a beam of radiation at the back of the throat for creating secondary waves that are captured and analyzed for forming a second set of reference spectroscopic data indicative of a vitamin C enhanced, silver-stained data of an uninfected state that is sent to a computer for storage and later comparison.

As given in block 314, at least one infected subjects are selected for spectroscopic examination for forming a set of reference spectroscopic data that is indicative of an infected state. For this purpose, a solution containing vitamin C is applied to the back of the throats of a set of infected subjects for forming pools of ascorbic acid within the layer of infected cells. After at least about three minutes, a solution containing metallic ions is applied to the back of the throat of at least one infected subjects for forming solid metal containing particles within the outer cellular layer of the throat, and a beam of electromagnetic radiation is directed at the outer-most cellular layer of the throats of the infected reference subjects for creating secondary waves from the vitamin C enhanced, silver-stained layer of infected cells. As given in block 316, the secondary waves are captured for forming a first set of spectroscopic data indicative of an infection, which data is sent to a computer for later comparison.

As given in block 318, a test subject is subjected to the vitamin C enhanced staining and excitation steps as outlined above for obtaining test spectroscopic data. As given in block 320, the test spectroscopic data is sent to the computer, and the data is compared with the uninfected and infected state spectra for determining if the test subject is infected, based on the observed similarities or differences between the test spectroscopic data the uninfected and infected state data sets.

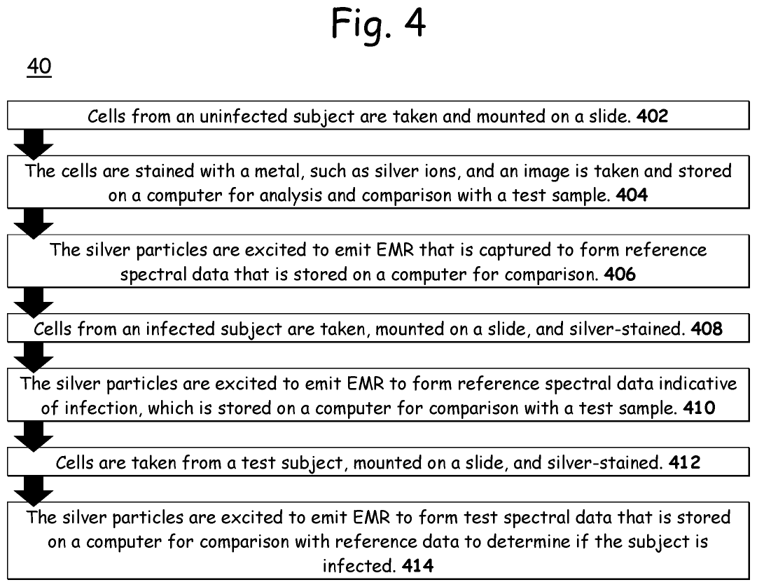

Now referring to FIG. 4, there is shown a flow chart of an in vitro method 40 for detecting the presence of a viral infection in a subject using emission-based methods. As given in block 402, a specimen of the cells from at least one uninfected subjects, which may be from the respiratory passages of the uninfected subject, is taken and placed on a slide. As given in block 404, the cells on the slide are stained with a substance comprised of metal, such as silver ions, for staining the cells, and a conventional microscopic image is taken and stored in a computer for later analysis and comparison with a test sample. For this purpose, the silver stain may be applied according to the Morozov method.

As given in block 406, the silver particles formed within the cells taken from the uninfected subjects are excited to emit electromagnetic radiation, which may be in the form of at least one of infrared, near infrared, visible, and ultraviolet light, which is captured for forming a set of reference spectral data of uninfected cells that is sent a computer for later comparison with a test sample.

As given in block 408, a specimen of cells from at least one infected subjects is taken and placed on a slide and silver stained. As given in block 410, after the infected cells have been stained, the silver particles are excited to emit electromagnetic radiation for forming a set of reference spectral data of infected cells. The set of data indicative of infection is sent to a computer for later comparison with a test sample.

As given in block 412, a specimen of cells is taken from a test subject and placed on a slide and silver stained. As given in block 414, the silver particles are excited to emit electromagnetic radiation for forming a set of test spectral data, which is sent to a computer for comparison with the uninfected subject and infected subject reference samples, for determining if the subject represented by the test spectral data is infected or not.

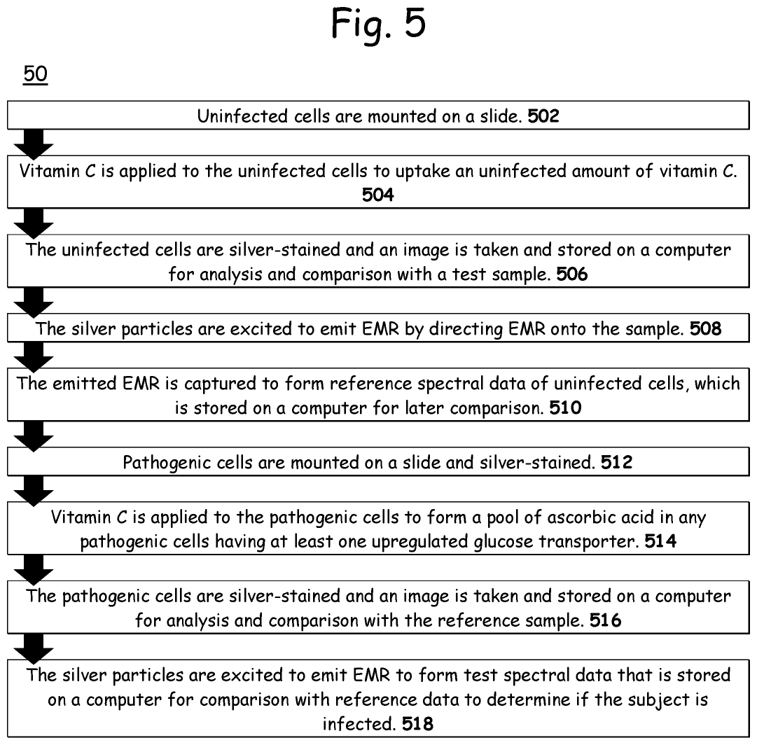

Now referring to FIG. 5, there is shown a block diagram of an in vitro method 50 for detecting the presence of a viral infection in a subject using vitamin C enhanced emission-based methods. As given in block 502, a specimen of the cells from at least one uninfected subject, which may be from the respiratory passages of the uninfected subject, is taken and placed on a slide. As given in block 504, vitamin C is applied to the slide for allowing the uninfected cells to uptake an amount of vitamin C. As given in block 506, the cells on the slide are silver stained and a conventional microscopic image is taken and stored on a computer for later analysis and comparison with a test sample. As given in block 508, the silver particles are excited to emit electromagnetic radiation by directing a beam of electromagnetic radiation upon the sample. As given in block 510, the emitted electromagnetic radiation is captured for forming a set of reference spectral data of uninfected cells that is sent to a computer for later comparison.

As given in block 512, a specimen of cells from at least one infected subject is taken and placed on a slide and silver stained. As given in block 514, vitamin C is applied to the slide for forming a pool of ascorbic acid within any pathogenic cells having at least one upregulated glucose transporter that may be present. As given in block 516, the cells are stained with silver for forming particulate matter within cells containing pools of ascorbic acid, and a conventional microscopic image of the particulate laden cells is taken and stored on a computer for later analysis and comparison with the reference sample. As given in block 518, the silver particles are excited to emit electromagnetic radiation, for forming a set of test spectral data that is sent to a computer for comparison with reference samples, for determining if the subject is infected.

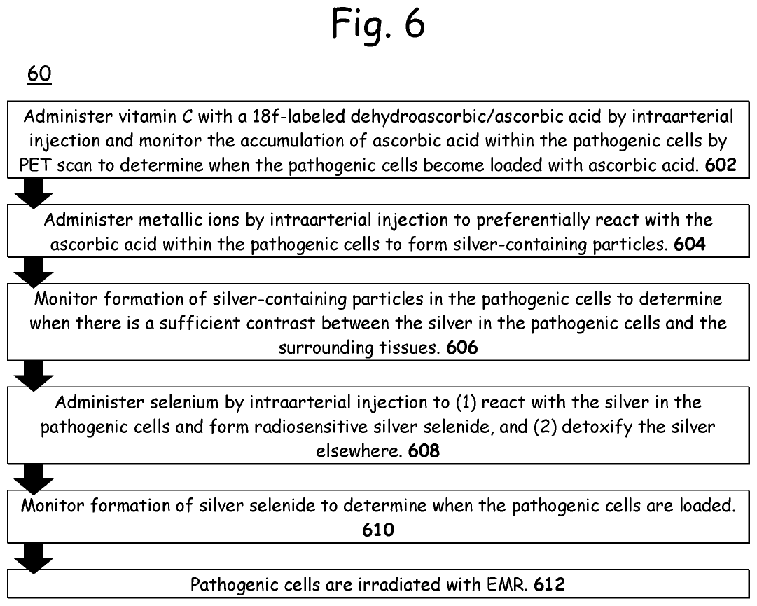

Now referring to FIG. 6, there is shown a block diagram of a first method 60 for the treatment of a patient suffering from proliferative disorder, wherein it is preferable that the pathogenic cells be destroyed by radiological energy. In some embodiments according to this aspect of the invention, the degree to which ascorbic acid can be accumulated within the pathogenic cells within a localized tumor is compared to surrounding tissues by the use of PET scan imaging techniques utilizing an intravenous-administered 18F-labeled radioactive tracer, for determining the degree of contrast in the possible accumulation of ascorbic acid within the localized tumor and surrounding tissues, for determining if there is enough contrast to carry out the treatment process, as given in block 602.

This embodiment of the invention is particularly useful in the treatment of proliferative diseases, such as, but not limited to cancer. The radioactive tracer can be an 18F-labeled form of glucose, such as 18F-labeled 2-fluoro-2deoxy-D-glucose (FDG), an 18F-labeled form of dehydroascorbic acid, or an 18F-labeled form of ascorbic acid or derivatives thereof, such as at least one of 6-deoxy-6-[18F] fluoro-L-ascorbic acid, 6-[18F]Fluoro-Dehydroascorbic Acid, and 6-Deoxy-6-[131I]iodo-L-ascorbic acid. In another embodiment, the radioactive tracer is a 14C-labeled form of at least one of glucose, dehydroascorbic acid, ascorbic acid, and derivatives thereof.

If the level of contrast is sufficient, vitamin C or derivatives along with a 18F-labeled form of either dehydroascorbic acid or ascorbic acid is administered to the patient by intraarterial injection as given in block 604, and the accumulation of ascorbic acid is monitored within the tumor by PET scan techniques for determining when the tumor becomes loaded with ascorbic acid in comparison to the surrounding tissues. After the tumor has reached a peak in the concentration of ascorbic acid, if the contrast between the tumor and surrounding tissues is not sufficient, the ascorbic acid within the surrounding uninfected tissues may be allowed to quickly dissipate until a better contrast between the tumor and the uninfected tissues has been reached.

After a comparatively high peak in the ascorbic acid level has been reached within the tumor, metallic ions are administered to the patient by intraarterial injection for preferentially reacting with the accumulated ascorbic acid within the tumor for forming solid particles containing silver therein. In one embodiment, the step as given in block 606 is used for either continuously or intermittently monitoring the formation of the silver-containing particles for determining the time at which a high degree of contrast is formed between the silver containing particles within the tumor and those of the surrounding tissues. The silver particles may be imaged, for example, by the use of x-ray imaging techniques or in the case where radioactive ions are used, the radioactivity may be measured in order to image the accumulation of the metal containing particles.

As given in block 608, selenium is administered to the patient by intraarterial injection for both (1) reacting with the particles of silver within the tumor for forming radiosensitive particles in the form of silver selenide, and (2) detoxifying the effects of silver in the remaining parts of the body. In one embodiment, the step as given in block 610 is used for continuously or intermittently monitoring the formation of the silver selenide for determining when the tumor becomes loaded with silver selenide. The formation of the silver selenide particles may be imaged, for example, by the use of x-ray imaging techniques.

As given in block 612, when a sufficient degree of contrast in the accumulation of silver selenide particles has been achieved between the tumor and the surrounding tissues, the tumor is irradiated with electromagnetic radiation for treating the subject. The electromagnetic radiation may be in the form of at least one of x-ray, microwave, and infrared radiation.

In one embodiment, before the administration of radiation, a treatment is administered to the patient to reduce the number of reactive oxidative species or reduce the track ionization density produced by the passage of radiation through both healthy cells and pathological cells. In one embodiment, the treatment includes administering a breathable gas containing a percentage of oxygen that is at least somewhat less than ambient air. In one embodiment the breathable gas has an oxygen concentration of from about ten percent to about fifteen percent.

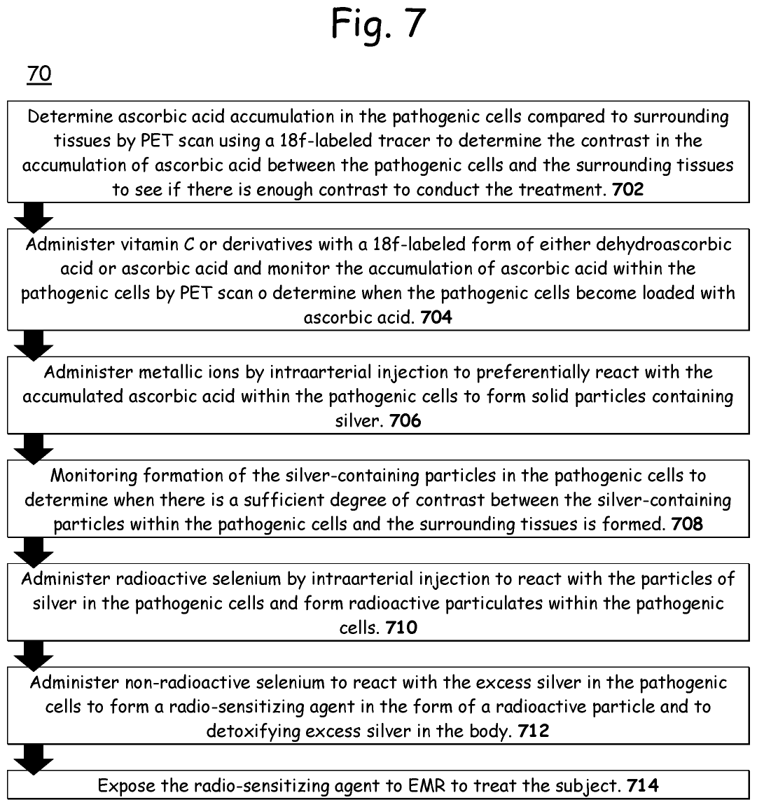

Now referring to FIG. 7, there is shown a block diagram of a second method for the treatment of a patient suffering from proliferative disorder, where it is preferable that the pathogenic cells be destroyed by radiological energy. In one embodiment, the method commences with the step as given in block 702, of determining the degree to which ascorbic acid can be accumulated within the pathogenic cells within a localized tumor, as compared to the surrounding tissues. When the level of contrast has been determined to be sufficient, vitamin C or derivatives along with a 18F-labeled form of either dehydroascorbic acid or ascorbic acid is administered, as given in block 704, by intraarterial injection into the patient, and the accumulation of ascorbic acid within the tumor is monitored by PET scan techniques to determine when the tumor becomes loaded with ascorbic acid, in comparison to the surrounding tissues.

As given in block 706, after a comparatively high peak in the ascorbic acid level has been reached within the tumor, metallic ions are administered to the patient by intraarterial injection to preferentially react with the accumulated ascorbic acid within the tumor to form solid particles containing silver. In one embodiment, step 708 is used to continuously or intermittently monitor the formation of the silver-containing particles, to determining when there is a sufficient degree of contrast between the silver-containing particles within the tumor and those of the surrounding tissues.

As given in block 710, a radioactive form of selenium is administered to the patient by intraarterial injection to react with the particles of silver within the tumor and form radioactive particulates within the tumor to treat the subject.

In one embodiment, after the radioactive form of selenium has been administered, a step as given in block 712 is performed, wherein a non-radioactive form of selenium is administered to the patient to react with the excess silver in the pathogenic cells and form a radio-sensitizing agent in the form of a radioactive particle, and to detoxify the excess silver in the body.

As given in block 714, the radioactive radio-sensitizing agent is exposed to electromagnetic radiation to treat the subject.

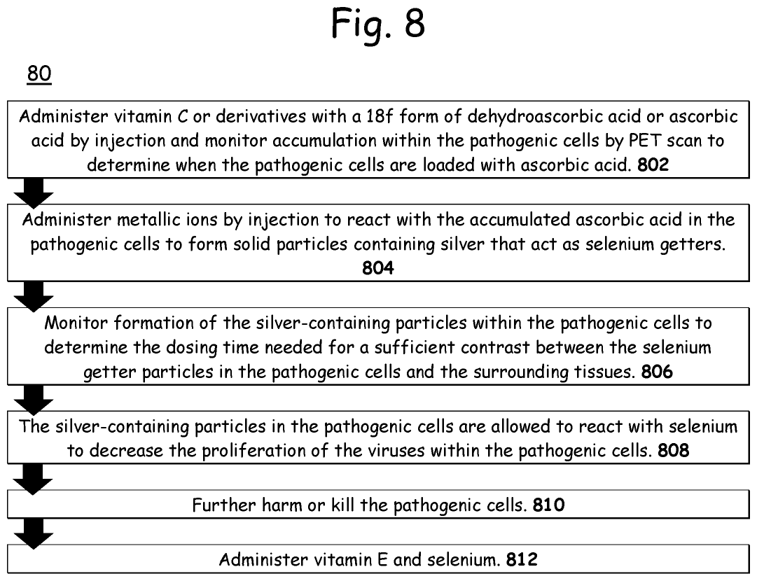

Now referring to FIG. 8, there is shown a block diagram of a method 80 for treating a patient suffering from either a proliferative disorder or an intracellular infection, wherein it is preferable that the pathogenicly-affected cells not be destroyed. As given in block 802, vitamin C or derivatives thereof are administered along with a 18F-labeled form of either dehydroascorbic acid or ascorbic acid by intraarterial injection into the patient, and monitoring the accumulation of ascorbic acid within the pathogenicly-affected cells by PET scan monitoring techniques to determine when the cells become loaded with ascorbic acid.

As given in block 804, metallic ions are administered to the patient by intravenous injection for preferentially reacting with the accumulated ascorbic acid within the tumor and for forming solid particles containing silver therein that act as a getter for selenium. In some embodiments, the formation of the silver-containing particles is continuously or intermittently monitored in the body of the patient, as given in block 806, to determine the dosing time needed for a sufficient degree of contrast to form between the selenium getter particles within the affected cells and the surrounding tissues. The silver-containing particles with the virally infected cells or the proliferative cells are allowed to react with selenium within the cells to decrease the proliferation of either the proliferative cells or the viruses within the cells.

In one embodiment, a step is undertaken as given in block 808, during the formation of the solid silver-containing particles. During this step the level of formation of the silver-containing particles within the tumor is continuously or intermittently monitored to determine when a peak level of silver-containing particles are formed. For this purpose, the formation of the particles may be determined by x-ray techniques.

In the case of virally infected and cells and proliferative cells in the form of smooth muscle, the silver-containing particles are allowed to react with the selenium within the pathogenic cells to decrease the rate of proliferation of either the smooth muscle cells or the cells containing viruses.

In those cases wherein it is desirable to either further harm the pathogenic cells, or to kill them, a fifth step may be undertaken, as given in block 810. After a leveling off in the formation of the solid silver-containing particles occurs, a sixth step is undertaken, as given in block 812, to reduce the toxic effects of the silver ions by administering both vitamin E and selenium to the patient.

As used herein, the phrase “at least one of A, B, and C” means all possible combinations of none or multiple instances of each of A, B, and C, but at least one A, or one B, or one C. For example, and without limitation: A×1, A×2+B×1, C×2, A×1+B×1+C×1, A×10+B×12+C×113. It does not mean A×0+B×0+C×0.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method for treating unwanted cells in a patient, the method comprising the steps of:
administering a first substance that is capable of forming a dehydroascorbic acid-like substance in the patient, thereby forming a pool of reactive molecules within the unwanted cells, and
administering a second substance for reacting with the pool of reactive molecules within the unwanted cells, thereby forming a product within the unwanted cells.

2. The method of claim 1 wherein the product comprises at least one of a sensitizing agent, a tracer agent, and a therapeutic agent.

3. The method of claim 1 wherein the second substance is comprised of at least one of:
metal ions that form the product with a cellular component of the unwanted cells, the cellular component in the form of at least one of sulfur-containing molecules and selenium-containing molecules.
radioactive halogen ions,
radioactive gold,
radioactive silver,
radioactive iodine ions,
nonradioactive halogen ions,
nonradioactive gold,
nonradioactive silver,
nonradioactive iodine ions, and
a constituent that does not pass across at least one of a blood-brain barrier and a blood-cerebral spinal fluid barrier.

4. The method of claim 1 further comprising the step of passing EMR through the product within the unwanted cells.

5. The method of claim 4 wherein the EMR comprises first filtered x-rays that selectively excite the product and thereby form second fluorescence x-rays that create radicals that kill the unwanted cells.

6. The method as in claim 4 further comprising the step of administering to the patient a treatment to reduce reactive oxidative species that are produced by passing EMR.

7. The method of claim 6 wherein the treatment comprises administering a breathable gas having an oxygen content of from about ten percent to about fifteen percent.

8. The method of claim 4 further wherein the second substance produces a sensitizing agent that sensitizes the unwanted cells to the EMR in cells that have a reduced oxygen tension.

9. The method of claim 1 further comprising the step of administering a GLUT-1 blocker to the patient after the pool of reactive molecules has been formed to prevent passage of glucose and vitamin C into the unwanted cells, thereby potentiating the unwanted cells to radiation-based therapies by entrapment of dehydroascorbic acid therein.

10. The method of claim 1 wherein the product lowers levels of glutathione within unwanted cells.

11. The method of claim 1 wherein the unwanted cells comprise at least one of pathologically-altered cells having upregulated glucose transporters, a pathological formation of proliferative cells, a cancerous tumor, proliferative smooth-muscle cells associated with hyperplasia of pulmonary artery smooth-muscle cells, cell types that produce an unwanted amount of fibrogenic materials, cells that have been invaded by an infective agent, and hyper-activated immune cells that cause hyper-inflammation.

12. The method of claim 1 wherein the unwanted cells are at least one of:
a pathological formation of proliferative cells,
a cancerous tumor,
proliferative smooth-muscle cells associated with hyperplasia of pulmonary artery smooth-muscle cells,
cell types that produce an unwanted amount of fibrogenic materials,
cells that have been invaded by an infective agent, and
hyper-activated immune cells that create a state of hyper-inflammation within the patient.

13. A method for classifying test cells, the method comprising the steps of:
applying first metallic ions to healthy cells,
irradiating the healthy cells,
recording properties of secondary emissions from the healthy cells,
applying second metallic ions to pathogenic cells,
irradiating the pathogenic cells,
recording properties of secondary emissions from the pathogenic cells,
applying third metallic ions to the test cells,
irradiating the test cells,
recording properties of secondary emissions from the test cells, and
comparing the properties of the test cells secondary emissions to the healthy cells secondary emissions and the pathogenic cells secondary emissions to determine whether the test cells are healthy or pathogenic.

14. The method of claim 13 wherein the first metallic ions, second metallic ions, and third metallic ions are silver.

15. The method of claim 13, further comprising the steps of:
applying a first substance that can form a metal-reducing agent within cells to the healthy cells prior to irradiating the healthy cells,
applying a second substance that can form a metal-reducing agent within cells to the pathogenic cells prior to irradiating the pathogenic cells, and
applying a third substance that can form a metal-reducing agent within cells to the test cells prior to irradiating the test cells.

16. The method of claim 15 wherein the first substance, second substance, and third substance are vitamin C.

17. The method of claim 13 wherein the applying and irradiating steps are performed in at least one of in-vitro, in-vivo, a subject's throat, and the subject's respiratory system.

18. The method of claim 13 wherein the pathogenic cells comprise at least one of virus-infected cells, proliferative cells, and cancer cells.
